(19) United States
(12) Plant Patent Application Publication
(10) Pub. No.: US 2006/0112472 P1
(43) Pub. Date: May 25, 2006

(54) Variety of Dahlia named 'Ham01'

(50) DAHLIA VARIABILIS; DAHLIA

(76) Inventor: Keith Hammett, Auckland (NZ)

Correspondence Address: FULWIDER PATTON LEE & UTECHT, LLP, 200 OCEANGATE, SUITE 1550, LONG BEACH, CA, 90802, US

(21) Appl. No.: 10/995,831

(22) Filed: Nov. 23, 2004

(51) Int. Cl. A01H 5/00

(52) U.S. Cl. PLT/321

(57) ABSTRACT

A new and distinct cultivar of Dahlia plant named ‘HAM01’ characterized by its distinctive dark green foliage and proportional foliage and blooms in relation to the overall size of the plant along with petals including a yellow to yellow-white midzone with white margin.

### BACKGROUND OF THE NEW PLANT

The present invention relates to a new and distinct cultivar of Dahlia, botanically known as Dahlia variabilis and further known by the varietal name ‘HAM01’. This new variety is also sold under the Breeder's reference name TWINKLE (Breeder's Ref. No. 71357-01). The new variety was invented in a controlled open pollination of selected parents by the inventor, Dr. Keith Hammett, in Massey, New Zealand. The parental cultivar is sold under the Breeder's reference name Tudor3 (Breeder's ID 71287/03) and is unpatented. The seeds were first collected in 2001 and seedlings selected in March of 2002. The cultivar was isolated by the inventor, Dr. Keith Hammett, in Massey, New Zealand, and selected primarily on the basis of foliage color and proportionality of the foliage and blooms in relation to the overall plant.

Following the seed selection, the cultivar was propagated vegetatively from cuttings at Massey, New Zealand in 2002. Continued observation of the vegetative divisions resulting from asexual reproduction of cuttings taken at Massey, New Zealand, having confirmed that the distinguishing features of this new cultivar came true, remain stable and are retained through successive propagation.

### SUMMARY OF THE INVENTION

Plants of the cultivar ‘HAM01’ have not been observed under all possible environmental and cultural conditions. The phenotype may vary somewhat with variations in environment such as temperature, light intensity, length of day, irrigation amount and frequency, and/or fertilizer rate without, however, any variance in genotype.

The following traits have been repeatedly observed and are determined to be among the unique characteristics of ‘HAM01’. These characteristics distinguish ‘HAM01’ as a new and distinct cultivar:

1. Darker green foliage than other dwarf Dahlia varieties;

2. Proportional foliage and inflorescence for a dwarf variety; and

3. Ray florets with an upper surface light yellow midzone near the base that transitions at a midpoint of the petal to yellow-white up to the outer distal edge and an upper surface margin that is white in color.

Plants of the new Dahlia variety can be compared to other plants of its genus. Compared to other dwarf Dahlia plants, the new Dahlia has a darker green foliage and, in contrast to the foliage and blooms of other dwarf Dahlia plants that are typically disproportionate in size to the plant, the foliage and blooms of the new Dahlia are more bonsai-like in that the foliage and blooms of the new variety are smaller in size and complement the dwarf nature of the plant. On the upper surface, the blooms of this new variety have a light yellow midzone near the base of the petal that transitions to a yellow-white midzone near the outer edge. The upper surface margin of each petal is white in color.

### BRIEF DESCRIPTION OF THE DRAWINGS

The invention is depicted in the accompanying colored photographs in which:

FIG. 1 shows a mature potted plant of the new variety;

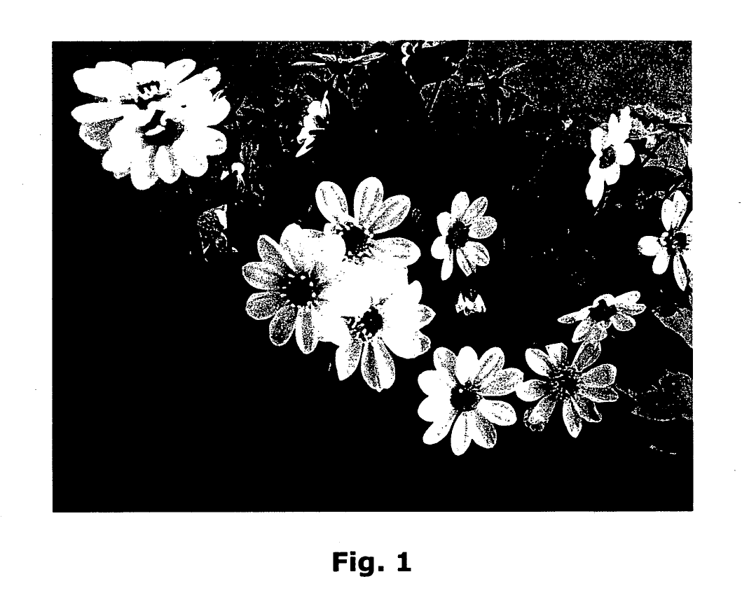

FIG. 2 shows a close up of a number of flowers of the plant of the new variety; and

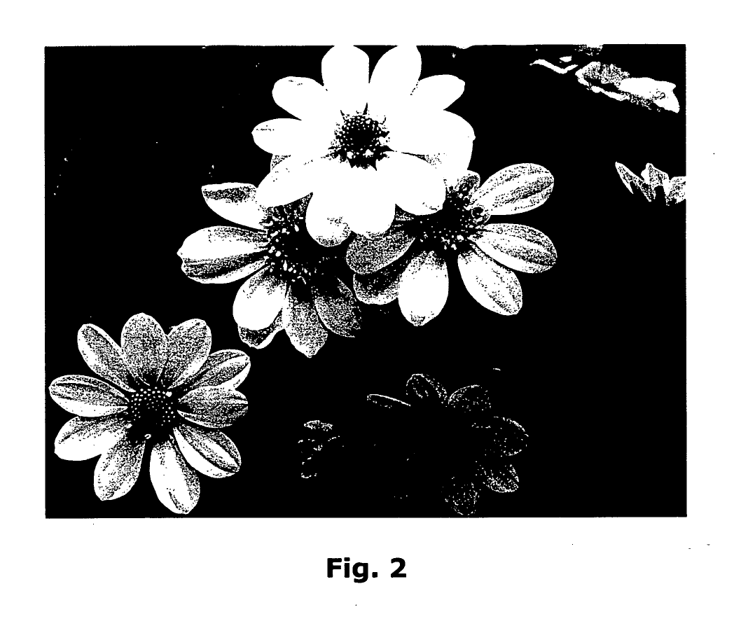

FIG. 3 is a close up view of the various parts of the new Dahlia variety including views of the upper and lower surfaces of representative foliage, views of the upper and lower surfaces of representative blooms, a view of a single bud, and a view of a bud on a stem.

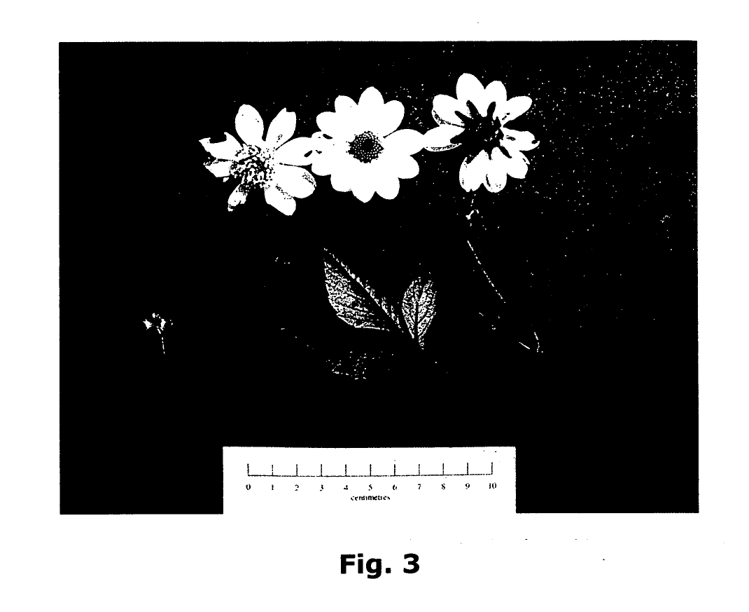

The photographs depict the overall appearance of the new Dahlia, showing the colors as true as it is reasonably possible to obtain in colored reproductions of this type. Colors in the photographs may differ slightly from the color values cited in the Detailed Botanical Description which more accurately describe the actual colors of the new Dahlia.

### DETAILED BOTANICAL DESCRIPTION OF THE NEW PLANT

The following detailed description sets forth characteristics of the new cultivar. The data which defines each characteristic was collected from asexual reproductions grown pots grown under plastic, and carried out by the breeder at Massey, New Zealand. The plant histories of 20 plants were taken on potted plants grown under plastic, in approximately October, 2004.

In the following table of comparative features, color references are made to The Royal Horticultural Society of London Colour Chart, 1995 Edition. The color values were determined on plant material grown in an open field at Massey, New Zealand.
Botanical name: Dahlia variabilis
Varietal name: ‘HAM01’
Parentage:
   Male.—Selected open pollinated.
   Female.—Tudor3 (Breeder's ID 71287/03).
The plant:
   Density.—Dense.
   Growth type.—Dwarf Compact.
   Growth rate.—Rapid, two months from cuttings to fully mature plant.
   Height.—300-350 mm.
   Width.—400 mm.
Leaf:
   Color.—Main — Yellow-green group RHS 147A.
   Color.—Vein — Brown-red group RHS 183A.
   Characteristics.—Ovate, generally unifoliate to trifoliate.
   Margin.—Serrated.
   Mean length.—60 mm.
   Mean width.—70 mm.
   Texture.—Glabrous.
Stem:
   Stem diameter.—3.5-4.5 mm.
Inflorescence:
   Length of flowering period.—Long overall with each flower individually lasting about 10 days.
   Time of flowering.—Early Spring through to mid Fall.
   Number of florets.—Less than 12 ray florets per capitulum.
   Flower size.—up to 50 mm.
   Fragrance of bloom.—None detected.
   Profile upper.—Flat.
   Inflorescence type.—Capitulum.
   Color midzone lower surface.—Yellow group RHS 4D.
   Color midzone upper surface.—Yellow Group RHS 4C.
   Color margin upper surface.—Yellow Group RHS 4C.
   Internal to disk florets.—Unopened Group RHS 174A Greyed orange group RHS 163B.
Flower bud:
   Shape.—Inflorescence bud, flat spherical.
   Mean length (mm) (at time of sepal separation).—11 mm.
   Mean width (mm) (at time of sepal separation).—7 mm.
   Color.—RHS 200B.
Petal:
   Number.—Ray floret up to 12.
   Petal size.—Ray floret small.
   Mean petal length.—Ray floret 18-21 mm.
   Mean petal width.—Ray floret 11-13 mm.
   Arrangement.—Ray florets overlap.
Pedicel:
   Prickles.—Single to double row of ray florets, central disc of disc florets.
   Mean length.—Stem from last pair of true leaves to involucral bracts.
Sepal:
   Type.—Involucral bracts.
   Number.—4-5.
Stamen:
   Anther color.—Yellow.
   Mean anther length (mm) (base to bend).—Not measurable.
Pistils:
   Mean style length (mm).—Not available.
   Style color.—Yellow.
Seed vessel characteristics:
   Type.—Achene.
   Size.—Small.
   Color.—Grey.
Development:
   Lasting quality.—Herbaceous perennial.
   Resistance to disease.—Very good.

### GENERAL CHARACTERISTICS

‘HAM01’ is primarily distinguished from other plants of the Dahlia genus by its darker green foliage, proportional foliage and bloom size for a dwarf variety of Dahlia, and petals with yellow to yellow-white midzone and white margins. ‘HAM01’ is a herbaceous perennial beginning its blooming season in early Spring and continuing until mid Fall with a consistent array of flowers throughout the season. Individual flowers last about 10 days. The foliage includes leaves of a generally ovate shape with serrated edges and have a unifoliate to tri-foliate arrangement. The foliage color is generally green RHS Color 147A with a vein of RHS Color 183A and darker than other varieties of the Dahlia genus, particularly the dwarf variety.

What is claimed is:

1. A new and distinct variety of Dahlia plant, substantially as shown and described.
